(19) United States
(12) Patent Application Publication
(10) Pub. No.: US 2022/0339022 A1
(43) Pub. Date: Oct. 27, 2022

(54) OSTOMY POUCH PROTECTOR

(71) Applicant: STOMALTERN INC., Quebec (CA)

(72) Inventor: Stéphanie WECHE, Quebec (CA)

(21) Appl. No.: 17/642,502

(22) PCT Filed: Sep. 10, 2020
(86) PCT No.: PCT/CA2020/051218

§ 371 Date: Mar. 11, 2022

Related U.S. Application Data
(60) Provisional application No. 62/900,010, filed on Sep. 13, 2019.

(51) Int. Cl. A61F 5/449; A61F 5/448

(52) U.S. Cl. CPC A61F 5/449; A61F 5/448

(57) ABSTRACT

The ostomy pouch protector is for protecting an ostomy pouch. It comprises an impermeable envelope defining an enclosure for receiving an ostomy pouch therein. The envelope comprises an ostomy opening allowing access into the enclosure, for allowing the ostomy pouch to be inserted within and removed from the enclosure and for allowing the ostomy pouch to connect to the ostomy through the ostomy opening. The envelope defines a rim about the ostomy opening. The envelope also comprises a skirt attached to the rim and extending away from the ostomy opening. A biasing attachment releasably attaches the envelope to the person and biases the skirt in sealing engagement against the abdomen of the person.

### FIELD OF THE INVENTION

The present invention relates to ostomies, and more particularly to an ostomy pouch protector for protecting an ostomy pouch, especially against water.

### BACKGROUND OF THE INVENTION

An ostomy is a surgical creation of an abdominal opening that enables a connection from an internal organ (such as the intestine) to the skin surface. In many cases, the purpose is to allow the elimination of bodily waste products, such as feces, through an extremity of the intestine that protrudes outside of the person's abdomen, when that person cannot eliminate normally through their anus. The opening of the ostomy on the skin is called a stoma. For some ostomies such as ileostomies and colostomies, the stoma effectively functions as an anus, however without any sphincter, which requires many people with these types of ostomies to wear an ostomy pouch to receive and hold bodily waste as it is evacuated. The ostomy pouch will be removed from time to time to empty it.

An ostomy may be necessary due to birth defects, cancer, inflammatory bowel disease, diverticulitis, incontinence and many other medical conditions. They are also necessary in cases of severe abdominal or pelvic trauma resulting from accidents or from injuries sustained during military service.

An ostomy consequently causes a change in the way urine or feces exit the body as a result of the surgical procedure: bodily waste is rerouted from its usual path into the ostomy pouch.

The ostomy pouch comprises a bag made of impermeable material that has a stoma opening fitted with a collar equipped with a wafer. A hole can be pierced through the wafer, inside the collar, of a size adapted to snugly receive the stoma therethrough, such that when the collar is applied against the abdomen, the stoma will extend through the hole made in the wafer and protrude within the pouch. The wafer is provided with an adhesive such that it will stick to the abdomen to both hold the ostomy pouch and create a seal about the stoma.

The pouch can be a one-piece or a two-piece system. In a one-piece system, the pouch is usually provided with a closable drainage opening at its bottom end to drain the pouch from the accumulated evacuated biological matter (feces, urine). In a two-piece system, the pouch is clipped onto the collar and may be removed from the collar, such that the collar will remain attached to the abdomen with the adhesive, while the pouch can be handled separately. The two-piece systems might not include a drainage opening, and the drainage may be made through the stoma opening.

Installing the ostomy pouch is a delicate undertaking that requires attention to detail and a few steps. Indeed, protectors or skin barriers in the form of pastes, gels, sheets or powders might be first applied to reduce the likelihood of infection or skin irritation. Then, the wafer opening needs to be made and adjusted to ensure a snug fit about the stoma. Finally, the bag itself needs to be installed by sticking the wafer to the abdomen. An incorrect or poorly adjusted installation can lead to the pouch leaking or accidentally falling off—both of which are undesirable.

When taking a shower, a bath or going to the pool, the ostomy pouch will usually be worn by the person to avoid the stoma from being subjected to the water, including the shower jet. Also, the stoma must not be subjected to certain types of soaps, shower gels or other body cleaning agents, so wearing the ostomy pouch is further desirable for that reason. When worn during a shower or bath, the pouch needs to be dried afterwards. It can be dried with a hairdryer using cold air, removed to dry it separately, or it can be changed for a new pouch. If the ostomy bag is not properly dried, it can result in the adhesive attaching the wafer to the abdomen being weakened to be point of not properly sealing the wafer to the skin or not properly attaching the wafer to the skin, or in water remaining between the bag and the abdomen or near the collar leading to the development of bacteria. Many ostomates consequently opt to keep their ostomy bag during a shower while trying to avoid getting the ostomy bag wet—a challenging undertaking.

### SUMMARY OF THE INVENTION

The present invention relates to an ostomy pouch protector for protecting an ostomy pouch comprising:
an impermeable envelope defining an enclosure for receiving an ostomy pouch therein, said envelope comprising:
   an ostomy opening allowing access into the enclosure for allowing the ostomy pouch to be inserted within and removed from the enclosure and for allowing the ostomy pouch to connect to the ostomy through the ostomy opening, the envelope defining a rim about the ostomy opening; and
   a skirt attached to the rim and extending away from the ostomy opening; and
a biasing attachment for releasably attaching the envelope to the person wearing the ostomy pouch protector and for biasing the skirt in sealing engagement against the abdomen of the person.

In one embodiment, the biasing attachment comprises an elastic strap, for attachment to the person's body.

In one embodiment, the elastic strap is attached to the rim and extends away from the rim between the envelope and the skirt.

In one embodiment, the elastic strap is a belt and extends around the person's waist.

In one embodiment, the biasing attachment comprises a garment that carries the envelope, with the ostomy opening facing inwardly of the garment when it is worn for engagement against the person's abdomen.

In one embodiment, the garment is one of a pair of pants or swim trunks.

The invention also relates to a protective impermeable envelope for protecting an ostomy pouch, the envelope defining an enclosure for receiving an ostomy pouch therein, the envelope comprising:
an ostomy opening allowing access into the enclosure, for allowing the ostomy pouch to connect to the ostomy through the ostomy opening, the envelope defining a rim about the ostomy opening; and
a skirt attached to the rim and extending away from the ostomy opening; and wherein the envelope is for releasable attachment with a biasing attachment to a person wearing the ostomy pouch, whereby the skirt is applied in sealing engagement against the abdomen of the person.

The invention further relates to a method of protecting an ostomy pouch from external fluids while it is installed on a person's abdomen, the method comprising:
providing an ostomy pouch protector comprising an impermeable envelope, the envelope defining an enclosure and comprising an ostomy opening in the envelope allowing access into the enclosure, the envelope defining a rim about the ostomy opening; the envelope further comprising a skirt attached to the rim and extending away from the ostomy opening; the ostomy pouch protector further comprising a biasing attachment attached to the envelope;
inserting the ostomy pouch within the enclosure such that the ostomy pouch connects to the ostomy through the ostomy opening;
releasably attaching the ostomy pouch protector to the person with the biasing attachment; and

biasing the skirt in sealing engagement against the abdomen of the person with the biasing attachment.

### DESCRIPTION OF THE DRAWINGS

### In the Annexed Drawings:

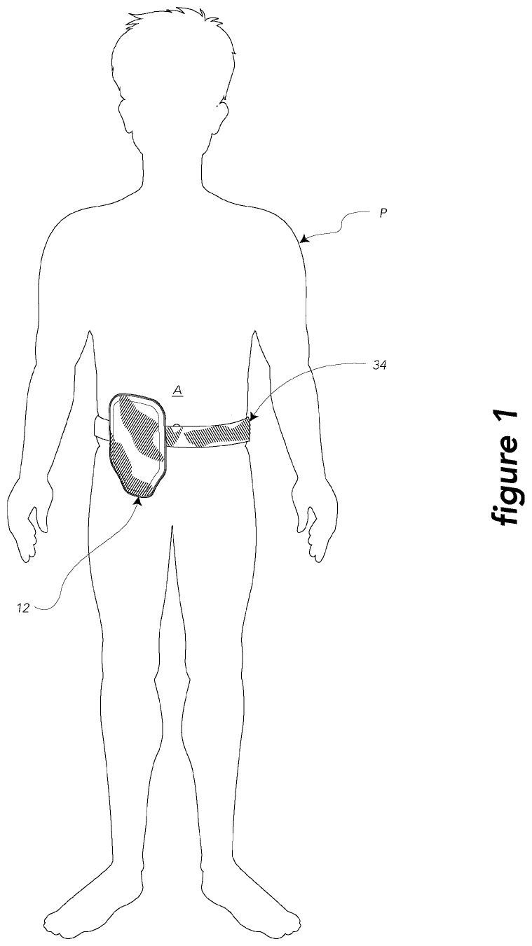

FIG. 1 is a front elevation of a person wearing an ostomy pouch protector according to a first embodiment of the invention;

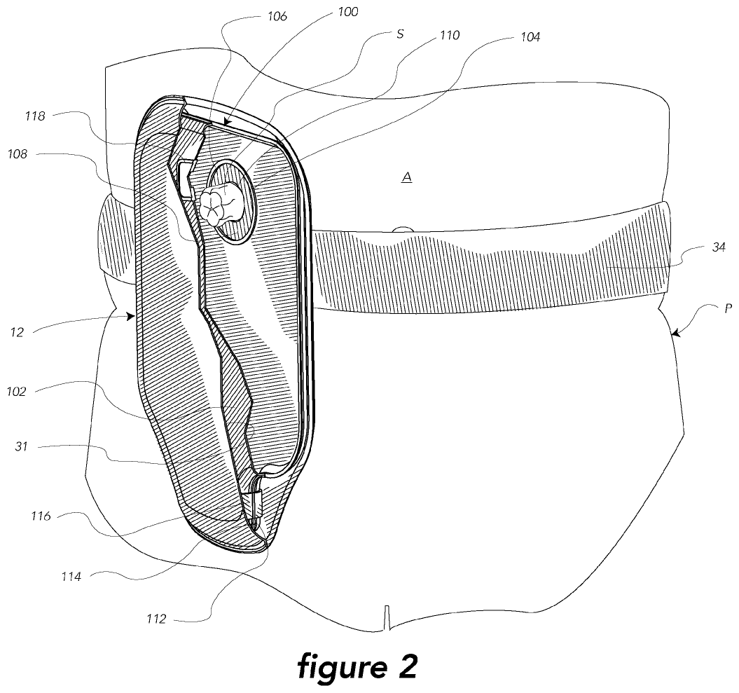

FIG. 2 is an enlarged partial front perspective view of the midriff portion of the person and the ostomy pouch protector of FIG. 1, with the ostomy pouch protector being partly broken to show the ostomy pouch contained therein and the stoma protruding out of the person's abdomen, through the wafer of the ostomy pouch and into the ostomy bag;

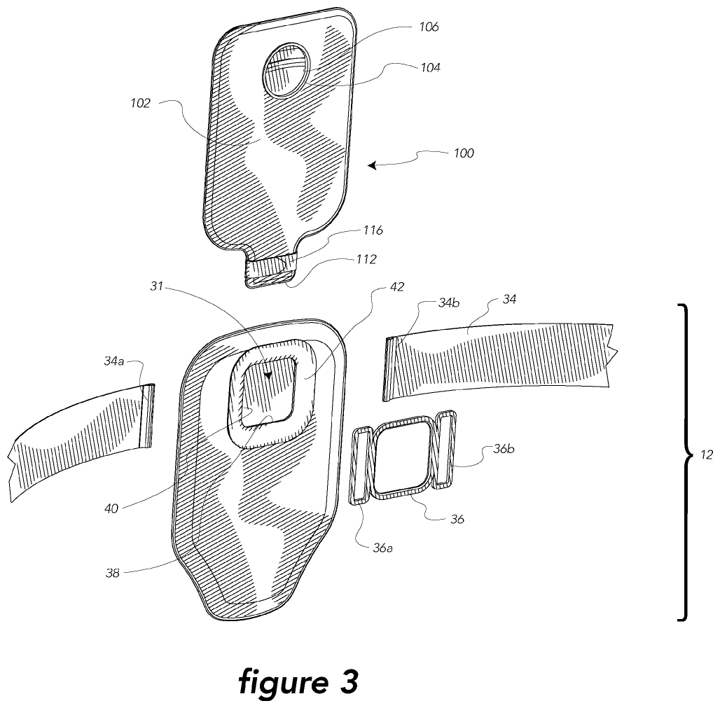

FIG. 3 is a partial rear perspective view of the ostomy pouch protector and ostomy pouch of FIG. 2, showing only part of the strap, and further showing in exploded view the ostomy pouch protector envelope, the strap, the strap collar and the ostomy pouch;

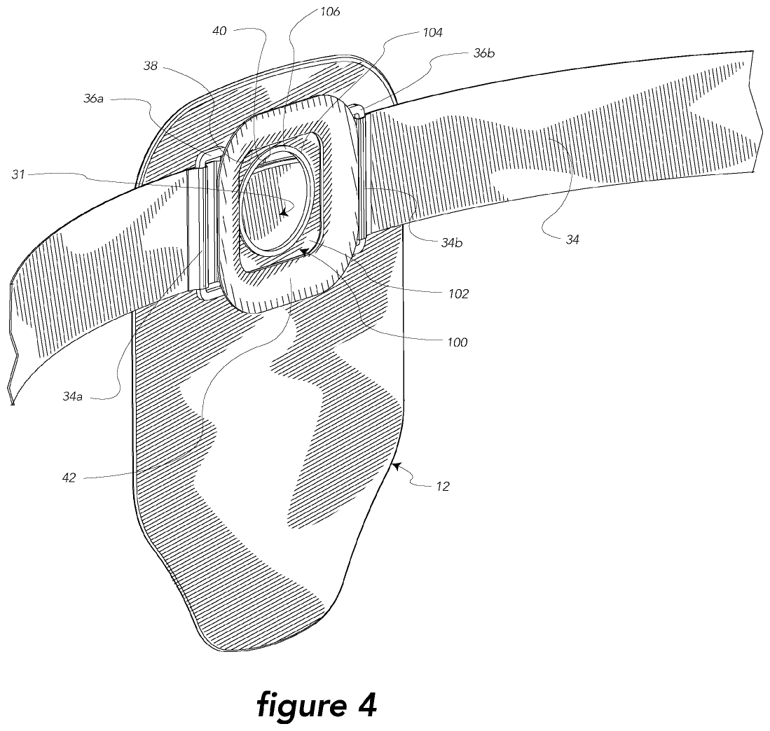

FIG. 4 is an enlarged rear partial perspective view of the ostomy pouch protector of FIG. 2, showing the ostomy pouch operatively inserted into the envelope, with the wafer of the ostomy pouch removed, and with the strap being only partly shown;

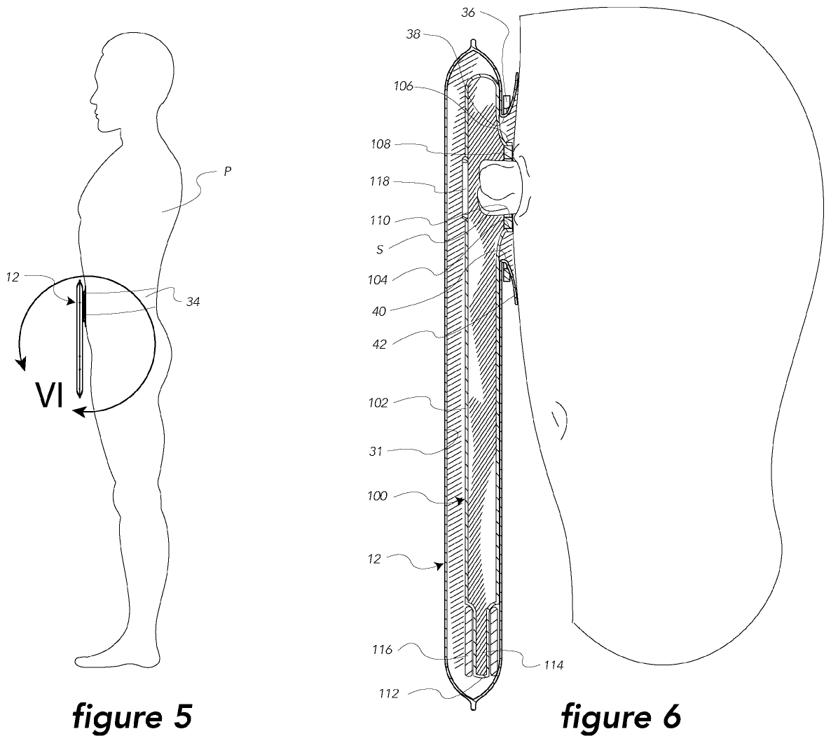

FIG. 5 is a side elevation, at a smaller scale, of the person and ostomy pouch protector of FIG. 1;

FIG. 6 is an enlarged view of the area circumscribed by circle VI in FIG. 5, with the strap being removed, and with the ostomy pouch protector and the ostomy pouch being shown in cross-section;

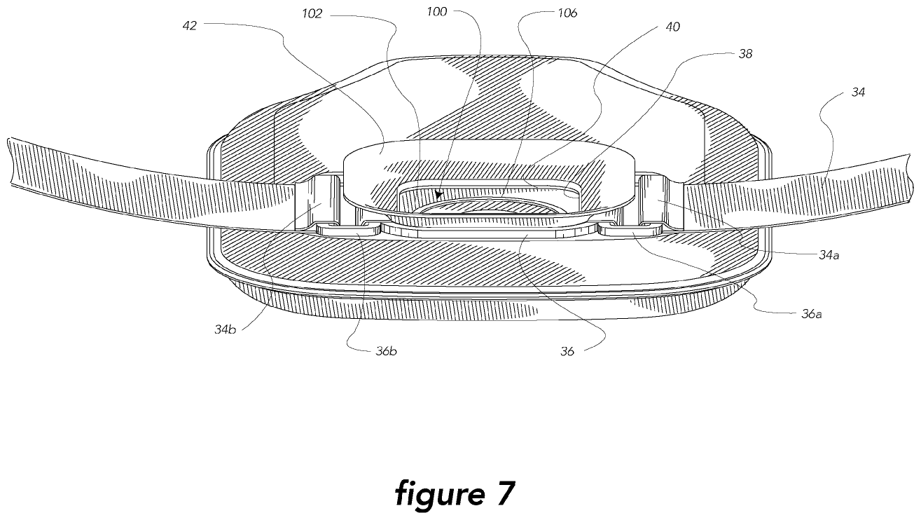

FIG. 7 is an enlarged top and rear partial perspective view of the ostomy pouch protector within which the ostomy pouch is inserted, as in FIG. 4, with the strap being only partly shown;

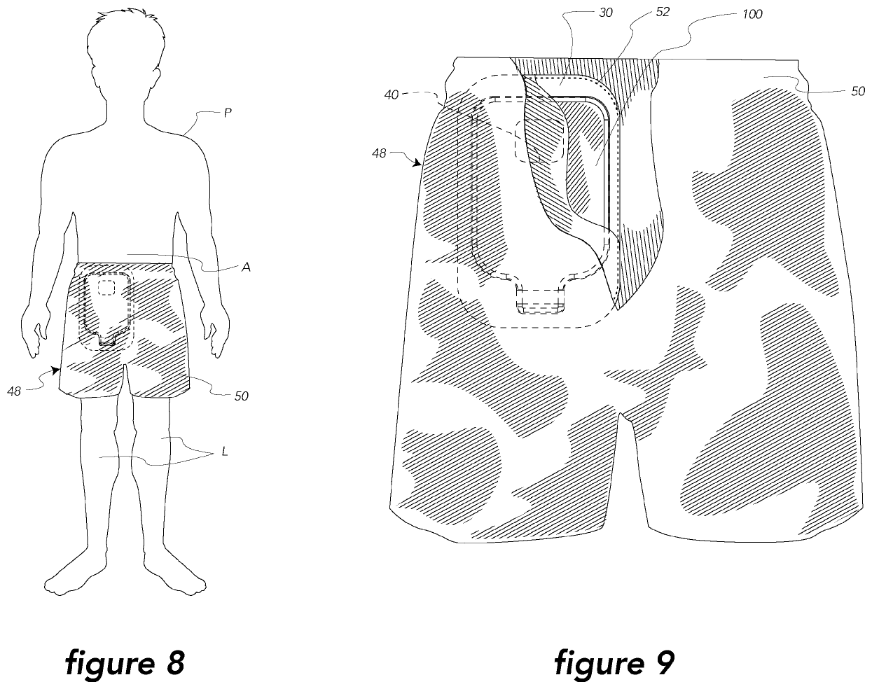

FIG. 8 is a front elevation of a person wearing an ostomy pouch protector according to another embodiment of the invention;

FIG. 9 is an enlarged front elevation of the ostomy pouch protector according to the embodiment of FIG. 8, with the garment and the envelope being partly broken;

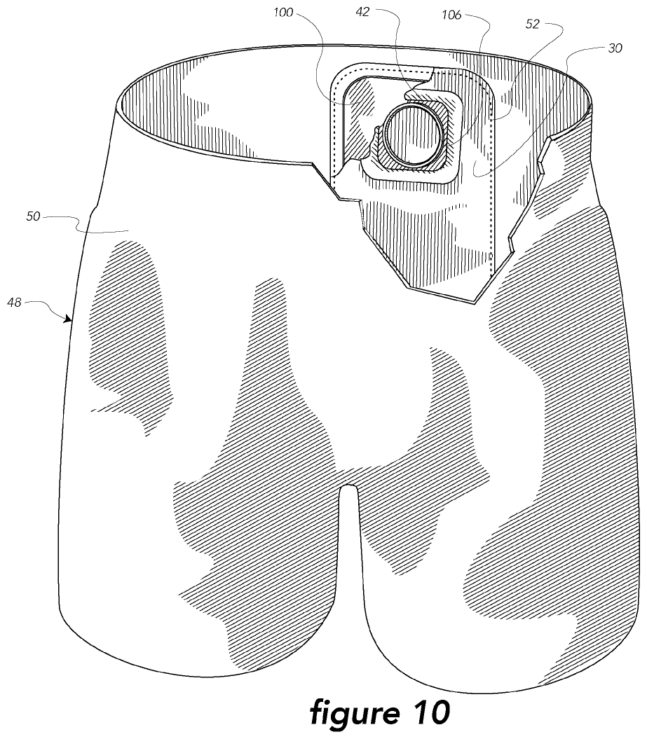

FIG. 10 is a rear perspective view of the ostomy pouch protector of FIG. 9 with the garment and ostomy pouch protector being partly broken and with the wafer of the ostomy pouch being removed; and

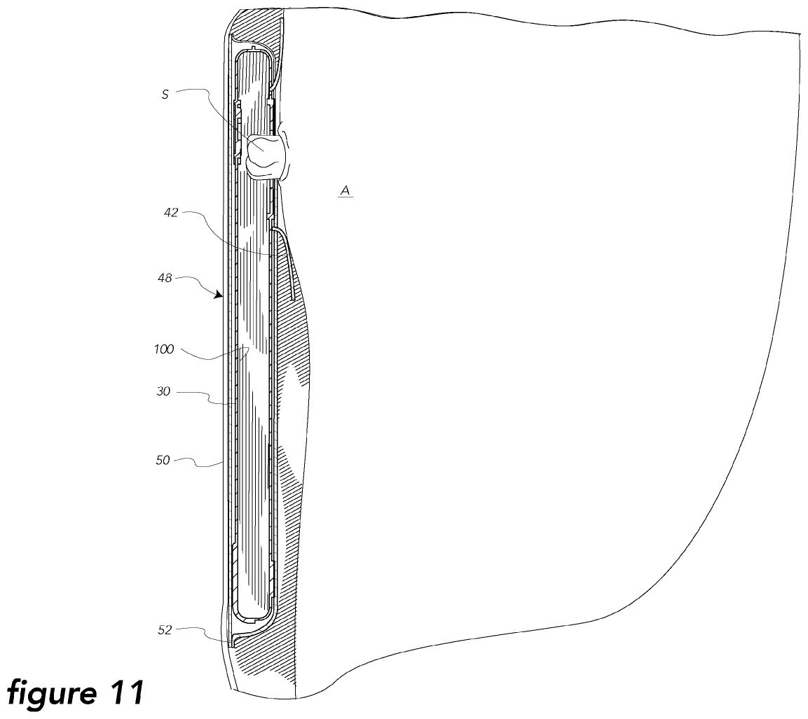

FIG. 11 is an enlarged partial side elevation of the midriff of the person wearing the ostomy pouch protector of FIG. 8, with the ostomy pouch protector being shown in cross-section.

### DETAILED DESCRIPTION OF THE EMBODIMENTS

FIGS. 1-7 show an ostomy pouch 100 installed on a person P (“the ostomate”) about its stoma S and covered with an ostomy pouch protector 12 according to the present invention. More particularly, the ostomy pouch 100 is of the conventional type and comprises a bag 102 provided with a stoma opening 104. A bag collar 106 is formed about opening 104. The collar 106 is equipped with a disc-shaped wafer 108 that is pierced near its center by the person to form a hole 110 to receive the stoma S therethrough. The collar 106 is applied against the person's abdomen A such that the wafer 108 will adhere to it by means of an adhesive that is provided on the wafer's rear face.

The bag 102 is oblong and comprises a bottom end 112 that has a drainage opening 114 fitted with a closable tail closure 116, for example in the form of a foldable Velcro™ seal clamp of known construction, or other equivalent closure. A filter 118, such as an active carbon filter, can be provided on bag 102.

Alternate ostomy bags of known construction can be used, including one-piece or two-piece systems.

According to the present invention, the ostomy pouch protector 12 is provided to protect the ostomy pouch 100 from water or other external threats, as detailed hereinafter.

Ostomy pouch protector 12 comprises an impermeable envelope 30 defining an enclosure 31 for receiving an ostomy pouch therein. Envelope 30 can be made of any suitable impermeable material that protects the ostomy pouch against fluids, including plastic, silicone, rubber, or the like.

Ostomy pouch protector 12 comprises a biasing attachment for releasably attaching the envelope 30 to person P. In the embodiment shown in FIGS. 1-7, the biasing attachment comprises an elastic strap 34 for attachment to the person's body. More particularly, strap 34 is in the form of a belt, and has two ends 34a, 34b sewn into loops for attachment to corresponding rigid oblong side rings 36a, 36b that are integrally linked to a strap collar 36. Strap 34 is preferably made of a stretchable elastic material.

Envelope 30 comprises an ostomy opening 38 allowing access into enclosure 31 and allowing the ostomy pouch 100 to connect to the stoma S through the ostomy opening. More particularly, ostomy opening 38 is wide enough to allow the bag collar 106 and the wafer to engage the person's abdomen. Envelope 30 defines a rim 40 that is the zone of envelope 30 located immediately about ostomy opening 31. Ostomy opening 38, circumscribed by rim 40, is consequently as wide as, or wider than, the bag collar 106 for allowing the proper engagement of bag collar 106 against the person's abdomen.

Envelope 30 also comprises a skirt 42 that extends away from rim 40. Skirt 42 extends outwardly away from opening 38, with rim 40, strap collar 36 and rings 36a, 36b being located between skirt 42 and the main body of envelope 30.

Envelope 30 is attached to strap collar 36 in a way to operatively allow installation and use of envelope 30 as detailed hereiafter. The attachment of envelope to strap collar 36 can simply rely on the resiliency of the material being used, e.g. such as that of silicone. If the envelope 30 and skirt 42 are resilient enough, it is possible to insert strap collar 36 between the skirt and the main body of envelope 30 and the intrinsic resiliency of the material will keep strap collar 36 in place. Or, a more positive attachment of strap collar to rim 40 could be envisioned, such as an adhesive, stitches or the like.

In use, ostomy pouch protector 12 can be installed on a person that has its ostomy pouch 100 already installed on them, with wafer 108 adhering to the person's abdomen. To proceed with the installation of pouch protector 12, the person will first slide the belt strap 34 around their legs and up until strap is approximately at waist level and the pouch protector 12 is located approximately at the level of ostomy pouch 10. Flexible strap 34 will stretch to accommodate the person's girth. Then, the person will slide ostomy bag 100 into the enclosure 31 of protector envelope 30 through opening 38. Since opening 38 is wider than collar 18, and since both ostomy bag 100 and envelope 30 are made of flexible material, this insertion is made easy. Bag 14 is entirely inserted into envelope 30 until rim 40 is positioned around the pouch's collar 106. Strap 34 is concurrently adjusted by sliding it on the abdomen to have it extend approximately horizontally around the abdomen.

In an alternate embodiment, a belt buckle (not shown) could be provided on belt strap 34 for attachment and removal of belt strap 34 around the person's abdomen. The strap would then not need to be elastic.

Flexible strap 34 will pull strap collar 36 towards the person's abdomen, which will apply pressure against skirt 42 that will be forced into a watertight sealing engagement against the person's body. Since the material of envelope 30 is suitable impermeable, such as a suitable plastic, silicone or rubber, it is further possible to have skirt 42 create a suction effect on the abdomen, further enhancing the sealing relationship with the abdomen and concurrently preventing the envelope rim 40 that circles wafer 108 from moving relative to wafer 108, i.e. preventing rim 40 from accidentally sliding against wafer 108 which might undesirably force wafer 108 to dislodge from engagement against the person's abdomen.

Water and other fluids will consequently be prevented from entering envelope 30 due to the watertight seal created by skirt 42, resulting in an efficient protection of both the ostomy pouch 10 and the peristomal area. This allows the person to take their shower or a bath, or even to be submerged in pool water, without the ostomy pouch becoming wet, in particular the delicate wafer seal 108 that can be affected and weakened by water to the point of losing its sealing and supporting properties; and without bacteria forming in the peristomal area.

When the person is done bathing, showering or swimming, and has dried, they can remove the pouch protector 12 very easily, by tangentially inserting a finger under skirt 42, between rim 40 and their abdomen, to let air in and break the suction effect; and then by sliding envelope 30 away from their abdomen, with the ostomy pouch 100 concurrently exiting enclosure 31 through opening 38; and finally by sliding the strap 34 down along their legs until the pouch protector 12 is entirely removed.

FIGS. 8-11 show another embodiment of an ostomy pouch protector 48 wherein the biasing attachment is in the form of a garment, and more particularly a midriff swimwear 50 that can be worn like swim trunks or shorts on the person's lower abdomen A and legs L. Garment 50 can be made for a male or female person. Garment 50 is preferably made of an elastic, stretchable material such that it will stretch when the person slides it on.

According to this embodiment, envelope 30 is carried by garment 50 and more particularly the outer peripheral edge 52 of envelope 30 is sewn to garment 50 (without the stiches piercing through into enclosure 31 of course). Envelope 30 is installed on the interior surface of garment 50 so as to face the person's abdomen when it is worn. Ostomy pouch 100 can be inserted and retrieved from envelope 30 as described in the first embodiment, with the exception that the garment is slid or removed from the person P as any other pair of shorts or swimming trunks might be donned or removed. The garment 50 carries the envelope 30 and biases the skirt 42 against the person's abdomen for creating a watertight seal with a suction effect of the skirt 42 on the abdomen, as described in the first embodiment.

In both described embodiments, the role of the biasing attachment is consequently both to carry the envelope and to bias the envelope skirt 42 against the person's abdomen. The skirt 42 will create the watertight seal to protect the ostomy pouch 100 and its wafer's attachment to the person's abdomen.

The skirt is advantageously made of the same material as the envelope, such as silicone, plastic or rubber. The skirt and the envelope proper could alternately be made of different suitable materials, i.e. the envelope proper in a first material, and the skirt in a second material. In all circumstances, the material(s) chosen for both the envelope and the skirt should be made of a material that is suitable according to a person skilled in the art, including in regard to being fluid tight, biocompatible, resistant, etc. . . . .

1-8. (canceled)

9. An ostomy pouch protector for protecting an ostomy pouch comprising:
an impermeable envelope defining an enclosure for receiving an ostomy pouch therein, said envelope comprising:
an ostomy opening allowing access into the enclosure for allowing the ostomy pouch to be inserted within and removed from the enclosure and for allowing the ostomy pouch to connect to the ostomy through the ostomy opening, the envelope defining a rim about the ostomy opening; and
a resiliently deformable skirt attached to the rim and extending away from the ostomy opening; and
a biasing attachment for releasably attaching the envelope to the person wearing the ostomy pouch protector and for biasing the skirt in sealing engagement against the abdomen of the person that creates a suction effect between the resilient skirt and the abdomen.

10. The ostomy pouch protector of claim 9, e biasing attachment comprises an elastic strap, for attachment to the person's body.

11. The ostomy pouch protector of claim 9, wherein the elastic strap is attached to the rim and extends away from the rim between the envelope and the skirt.

12. The ostomy pouch protector of claim 11, wherein the elastic strap is a belt and extends around the person's waist.

13. The ostomy pouch protector of claim 9, wherein the biasing attachment comprises a garment that carries the envelope, with the ostomy opening facing inwardly of the garment when it is worn for engagement against the person's abdomen.

14. The ostomy pouch protector of claim 13, wherein the garment is one of a pair of pants or swim trunks.

15. A protective impermeable envelope for protecting an ostomy pouch, the envelope defining an enclosure for receiving an ostomy pouch therein, the envelope comprising:
an ostomy opening allowing access into the enclosure, for allowing the ostomy pouch to connect to the ostomy through the ostomy opening, the envelope defining a rim about the ostomy opening; and
a resiliently deformable skirt attached to the rim and extending away from the ostomy opening; and
wherein the envelope is for releasable attachment with a biasing attachment to a person wearing the ostomy pouch, whereby the skirt is applied in sealing engagement against the abdomen of the person to create a suction effect between the resiliently deformable skirt and the abdomen.

16. A method of protecting an ostomy pouch from external fluids while it is installed on a person's abdomen, the method comprising:
providing an ostomy pouch protector comprising an impermeable envelope, the envelope defining an enclosure and comprising an ostomy opening in the envelope allowing access into the enclosure, the envelope defining a rim about the ostomy opening; the envelope further comprising a resiliently deformable skirt attached to the rim and extending away from the ostomy opening; the ostomy pouch protector further comprising a biasing attachment attached to the envelope;
inserting the ostomy pouch within the enclosure such that the ostomy pouch connects to the ostomy through the ostomy opening;
releasably attaching the ostomy pouch protector to the person with the biasing attachment; and
biasing the resilient skirt in sealing engagement against the abdomen of the person with the biasing attachment such that a suction effect is created between the resiliently deformable skirt and the abdomen.
